(19) United States
(12) Patent Application Publication
(10) Pub. No.: US 2019/0111001 A1
(43) Pub. Date: Apr. 18, 2019

(54) MICROPARTICULATE SYSTEM FOR COLONIC DRUG DELIVERY

(71) Applicants: Hanping FENG, Ellicott City, MD (US); Hua YU, Columbia, MD (US); Stephen HOAG, Catonsville, MD (US); Bowen JIANG, Columbia, MD (US)

(72) Inventors: Hanping FENG, Ellicott City, MD (US); Hua YU, Columbia, MD (US); Stephen HOAG, Catonsville, MD (US); Bowen JIANG, Columbia, MD (US)

(73) Assignee: University of Maryland, Baltimore, Baltimore, MD (US)

(21) Appl. No.: 16/089,564

(22) PCT Filed: Mar. 30, 2017
(86) PCT No.: PCT/US17/24990

§ 371 Date: Sep. 28, 2018

Related U.S. Application Data
(60) Provisional application No. 62/315,485, filed on Mar. 30, 2016.

(51) Int. Cl. A61K 9/50; C07K 16/12; A61K 39/40; A61P 31/04

(52) U.S. Cl. CPC A61K 9/5078; A61K 9/5026; C07K 16/1282; A61K 2039/505; A61P 31/04; C07K 2317/30; A61K 39/40

(57) ABSTRACT

A delivery system that allows for targeted released of therapeutics in selected regions of the GI tract is provided. The delivery system is adapted to deliver a certain type of therapeutic, such as a protein-based therapeutic, to a certain area of the GI tract, such the colon of a subject. The delivery vehicle comprises a microbead coated with layers that include: a subcoat layer and a therapeutic agent layer. The delivery vehicles may also include an enteric coating layer and/or a sustained release layer.

### STATEMENT OF FEDERALLY SPONSORED RESEARCH AND DEVELOPMENT

This invention was made with government support under Grant Number AI109776 awarded by the National Institutes of Health. The government has certain rights in the invention.

### BACKGROUND

Since the 1980s, protein therapeutics have become increasingly important tools in the treatment and prevention of various diseases and conditions in humans, with more than 240 protein or peptide therapeutics now available, by some estimates [1]. The advantages of protein therapeutics over small molecules include high specificity and potency, lower potential of adverse effects, and longer half-life [2]. However, most protein therapeutics must be administrated parenterally due to acidic degradation in the upper GI tract, enzymatic digestion, and low permeability through epithelial cells, among other barriers.

The development of improved means for oral delivery of proteins would provide clinicians with additional treatment options and help spur further development of protein therapeutics for difficult to treat diseases. For example, local delivery of protein therapeutics to the colon targets treatment at the sites of disease or infection, and avoids the need to flood the blood stream with a high concentration of therapeutics.

Inflammatory Bowel Disease (IBD) is an exemplary target disease for oral delivery of protein treatments. IBD includes inflammation of different sections of the gastro-intestinal (GI) tract, caused by colonized bacteria and locally secreted immune defense molecules. A site-specific delivery system of protein therapeutics might target those local entities, without prerequisite of absorption into blood. Similarly, Clostridium difficile infection (CDI) is a colon infection caused by Clostridium difficile bacteria. C. difficile produces two enterotoxins, toxin A (TcdA) and toxin B (TcdB), which trigger disease symptoms including antibiotic-associated diarrhea and life-threatening pseudomembranous colitis [3,4]. The current treatment of CDI includes oral administration of metronidazole, vancomycin and fidaxomicin; however, these drugs are insufficient for acute and recurrent CDI. Protein therapeutics targeted to the colon might serve to bind and block the activity of TcdA and TcdB in the patient. Additional diseases in which improved means of oral delivery might be important include ulcerative colitis and Crohn's Disease.

The present invention is directed to novel systems for colonic delivery of therapeutic agent, such as drugs and protein therapeutics, among other important goals.

### BRIEF SUMMARY

The present invention can be generally characterized as being drawn to vehicles for delivery of a therapeutic agent to the GI tract of a subject, where the delivery vehicle comprises a microbead coated with layers that include: a subcoat layer and a therapeutic agent layer. The delivery vehicles of the invention will also often include an enteric coating layer and/or a sustained release layer. The invention is further drawn to methods for preparing the delivery vehicles and to methods of treating or preventing diseases and conditions using the delivery vehicles.

Thus, and in a first embodiment, the invention is drawn to delivery vehicles comprising a microbead coated with a subcoat layer, a therapeutic agent layer, and an enteric coating layer. In certain aspects of the embodiment, the microbead is further coated with a sustained release layer.

The microbeads of the first embodiment comprise one or more physiologically inert substances. The subcoat layer substantially coats the microbead. The therapeutic agent layer substantially coats the subcoat layer and comprises a therapeutic agent and one or more excipients. The enteric coating layer substantially coats the therapeutic agent layer and comprises a pH-resistant composition. When present, the sustained release layer substantially coats the therapeutic agent layer and the enteric coating layer substantially coats the sustained release layer.

In a second embodiment, the invention is drawn to methods of preparing the delivery vehicles of the invention. In one aspect, the invention is drawn to methods of preparing a delivery vehicle that comprises a microbead coated with a subcoat layer, a therapeutic agent layer, and an enteric coating layer. In this aspect, the method comprises: (a) providing a microbead comprising one or more physiologically inert substances; (b) applying a subcoat layer to the microbead of (a), wherein the subcoat layer substantially coats the microbead; (c) applying a therapeutic agent layer to the microbead produced in (b), wherein the therapeutic agent layer substantially coats the subcoat layer, and wherein the therapeutic agent layer comprises a therapeutic agent and one or more excipients; and (d) applying an enteric coating layer to the microbead produced in (c), wherein the enteric coating layer substantially coats the therapeutic agent layer, and wherein the enteric coating layer comprises a pH-resistant composition.

In another aspect, the invention is drawn to methods of preparing a delivery vehicle that comprises a microbead coated with a subcoat layer, a therapeutic agent layer, a sustained release layer, and an enteric coating layer. In this aspect, the method comprises: (a) providing a microbead comprising one or more physiologically inert substances; (b) applying a subcoat layer to the microbead of (a), wherein the subcoat layer substantially coats the microbead; (c) applying a therapeutic agent layer to the microbead produced in (b), wherein the therapeutic agent layer substantially coats the subcoat layer, and wherein the therapeutic agent layer comprises a therapeutic agent and an excipient; (d) applying a sustained release layer to the microbead produced in (c), wherein the sustained release layer substantially coats the therapeutic agent layer; and (e) applying an enteric coating layer to the microbead produced in (d), wherein the enteric coating layer substantially coats the sustained release layer, and wherein the enteric coating layer comprises a pH-resistant composition.

In each of the relevant embodiments and aspects of the invention, the microbeads may comprise one or more of a sugar, a starch, microcrystalline cellulose (MCC), a biodegradable polymer, sodium phosphate, and calcium phosphate, or a mixture of two or more thereof. When the microbeads comprise a sugar, the sugar may be selected from the group consisting of lactose, sucrose, mannitol, trehalose, maltodextrin, dextrose, fructose and a polysaccharide, and a mixture of two or more thereof. When the microbeads comprise a biodegradable polymer, the biodegradable polymer may be poly(lactic-co-glycolic acid) (PLGA) or a copolymer of L-lactic acid and glycolic acid. The microbeads may also comprise a medicament. The microbeads may have an average particle diameter of between 1 and 1000 μm.

In each of the relevant embodiments and aspects of the invention, the subcoat layer may comprise one or more of ammonium alginate, cellaburate, chitosan, colophony, copovidone, ethylene glycol and vinyl alcohol grafted copolymer, gelatin, hydroxypropyl cellulose, hypromellose, hypromellose acetate succinate, polymethacrylate, poly(methyl vinyl ether/maleic anhydride), polyvinyl acetate dispersion, polyvinyl acetate phthalate, polyvinyl alcohol, polyvinylpyrrolidone (PVP), povidone, pullulan, pyroxylin, and shellac. The subcoat layer may provide an amount of weight gain to the microbeads of between about 0.1 and 5%. In a selected aspect, the subcoat layer comprises PVP and imparts about a 1% weight gain to the microbeads.

In each of the relevant embodiments and aspects of the invention, the therapeutic agent may comprise one or more of a protein, a peptide, an antibody, an antiviral, an antifungal, an antibiotic, an anticancer agent, an analgesic, an anticoagulant, an antidepressant, an antiepileptic, an antipsychotic, and a sedative. In a selected aspect, the therapeutic agent is an antibody. In a further selected aspect, the therapeutic agent is an antibody or fragment thereof having binding specificity for C. difficile toxin A and/or toxin B. The excipient may comprise one or more of polyvinylpyrrolidone (PVP), polyethylene glycol (PEG), polyvinyl acetate (PVA), hydroxypropyl cellulose (HPC), sucrose, trelose, acacia, tragacanth, gelatin, starch, pregelatinized starch, alginic acid, cellulose, methyl cellulose, ethyl cellulose, sodium carboxy methyl cellulose, polymethacrylate, asparagine, dextran, glycine, inulin, lactose, anhydrous lactose, monohydrate, mannitol, raffinose, and trehalose. The therapeutic agent layer may provide an amount of weight gain to the microbeads comprising the subcoat layer of between about 10 and 400%.

In each of the relevant embodiments and aspects of the invention, the pH-resistant composition of the enteric coating layer may comprise one or more of inulin, shellac, methacrylated inulin, pectin, chitosan, Eudragit® FS 30D, Eudragit® S 100 and Eudragit® S12,5. The pH-resistant composition of the enteric coating layer may further comprise a plasticizer. The enteric coating layer may provide an amount of weight gain to the microbeads comprising the subcoat layer and the therapeutic agent layer of between about 20 and 30%.

In each of the relevant embodiments and aspects of the invention, when present the sustained release layer may comprise one or more of acacia, agar, alginic acid, aliphatic polyester, calcium alginate, carbomer, carrageenan, cellaburate, cellulose acetate, ceratonia, copovidone, gellan gum, guar gum, hydroxyethylmethyl cellulose, hydroxypropyl betadex, hydroxypropyl cellulose, hypromellose, methylcellulose, polycarbophil, poly(DL-lactic acid), polymethacrylate, polyoxylglyceride, polyvinyl acetate dispersion, shellac, sodium alginate, starch modified, xanthan gum, zein, Eudragit® RL, Eudragit® RL 30D, Eudragit® RL PO, Eudragit® RL 100, Eudragit® RL 12,5, Eudragit® RS 30 D, Eudragit® RS PO, Eudragit® RS 100, Eudragit® RS 12,5, Eudragit® NE 30 D, Eudragit® NE 40 D, and Eudragit® NM 30 D. The sustained release layer may provide an amount of weight gain to the microbeads comprising the subcoat layer and the therapeutic agent layer of between about 5 and 15%.

In each of the relevant embodiments and aspects of the invention, the layers may be applied using spray coating, dip coating, powder coating, hot melt-extrusion, and/or spray drying.

In a third embodiment, the invention is drawn to methods of treating a disease or a condition in a subject. The methods comprise administering a therapeutically-effective amount of a delivery vehicle defined herein to a subject in need thereof, thereby treating the disease or condition in the subject. In one aspect, the disease or condition is one or more of an intestinal inflammatory disease, an autoimmune disease, an inflammatory bowel disease (IBD), celiac disease, irritable bowel syndrome, a bacterial infection, a viral infection, a fungal infection, Clostridium difficile infection (CDI), and a gastric intestinal tract malignancy. In a related aspect, the disease or condition is inflammatory bowel disease (IBD) and the therapeutic agent is an antibody that blocks the activity of secreted immune defense molecules. In another aspect, the disease or condition is Clostridium difficile infection (CDI) and the therapeutic agent is an antibody or fragment thereof having binding specificity for C. difficile toxin A (TcdA) and/or toxin B (TcdB).

In a fourth embodiment, the invention is drawn to methods of treating or preventing a disease symptom induced by C. difficile in a subject comprising administering a therapeutically-effective amount of a delivery vehicle defined herein to a subject having C. difficile infection or a risk of developing C. difficile infection.

In a fifth embodiment, the invention is drawn to methods of neutralizing C. difficile toxin TcdA and/or TcdB in a subject infected by C. difficile comprising administering a therapeutically-effective amount of a delivery vehicle defined herein to a subject having C. difficile infection. The neutralizing may be partial or full neutralization.

In a sixth embodiment, the invention is drawn to methods of treating or preventing C. difficile infection in a subject comprising administering a therapeutically-effective amount of a delivery vehicle defined herein to a subject having C. difficile infection or a risk of developing C. difficile infection.

In each of the third through sixth embodiments, the delivery vehicle may be in a pharmaceutical formulation comprising the delivery vehicle and a pharmaceutically acceptable carrier or diluent.

In each of the third through sixth embodiments, the therapeutically-effective amount of the delivery vehicle may be between 10 ug/kg and 100 mg/kg of the agent per body weight of the subject.

In each of the third through sixth embodiments, the delivery vehicle may be administered to the subject orally, parenterally or rectally.

In each of the third through sixth embodiments, the therapeutic agent may be an antibody. In one aspect, the therapeutic agent is an antibody or fragment thereof having binding specificity for C. difficile toxin A and/or toxin B.

The foregoing has outlined rather broadly the features and technical advantages of the present invention in order that the detailed description of the invention that follows may be better understood. Additional features and advantages of the invention will be described herein, which form the subject of the claims of the invention. It should be appreciated by those skilled in the art that any conception and specific embodiment disclosed herein may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that any description, figure, example, etc. is provided for the purpose of illustration and description only and is by no means intended to define the limits the invention.

### BRIEF DESCRIPTION OF DRAWINGS

FIG. 1. Structure of an exemplary colonic delivery vehicle. A microbead inner core is layered in successive order with a subcoat layer and a therapeutic agent layer. The microbeads may also optionally include a sustained release layer and/or an enteric coating layer.

FIGS. 2A-2B. BSA aggregation. (FIG. 2A) Size-exclusion chromatograms (detection at 280 nm) of commercial BSA dissolved in HPLC grade water, BSA mixed with PVP30 in the spray solution, and reconstituted BSA right after spray coating, after one month storage. (FIG. 2B) Size-exclusion chromatograms of reconstituted samples from BSA beads stored at 4° C., 25° C. and 40° C.

FIGS. 3A-3B. Secondary structure change in BSA. (FIG. 3A) Circular dichroism spectra of commercial BSA in HPLC grade water, mixed with PVP30 in spray solution, and reconstituted BSA after spray coating. (FIG. 3B) Secondary structure components of BSA right after spray coating and after one month storage at 4° C., 25° C. and 40° C.

FIGS. 4A-4B. (FIG. 4A) Secondary derivative UV-vis spectra of commercial BSA, mixed with PVP30 in spray solution, and reconstituted BSA after spray coating. (FIG. 4B) Secondary derivative UV-vis spectra of BSA right after one month storage at 4° C., 25° C. and 40° C.

FIG. 5. In vitro release of BSA from the colonic delivery vehicle with variable coating thickness.

FIG. 6. In vitro release of IgG-ABAB from enteric coated microbeads.

FIG. 7. In vivo release of IgG-ABAB from enteric coated microbeads in mouse GI tracts.

FIGS. 8A-8C. These figures provide the results on the analysis of mannitol microbeads (NONPAREIL®-108) sub-coated with 1% PVP 30 and then layered with one of the following five therapeutic agent layers: Formulation 1 (F1): IgG (10mg/mL) in water; Formulation 2 (F2): IgG +PVP (2.5%); Formulation 3 (F3): IgG +PVP (2.5%) +Trehalose (100% w/w of IgG); Formulation 4 (F4): IgG +PVP (2.5%) +Sucrose (100% w/w of IgG); Formulation 5 (F5): IgG +PVP (2.5%) +Arginine (100% w/w of IgG). FIG. 8A provides the results of turbidity analysis on reconstituted solutions of the different beads, measured using a UV-vis spectrometer at a wavelength of 340 nm. FIG. 8B provides protein recovery as measured by UV-vis spectroscopy at 280 nm using an extinction coefficient of 1.36. FIG. 8C provides monomer percentages of IgG in the reconstituted solutions as measured by size-exclusion FPLC at 280 nm after filtration with a 0.22 micron polyethersulfone (PES) filter unit.

### DETAILED DESCRIPTION

I. Definitions

Unless otherwise noted, technical terms are used according to conventional usage. Definitions of common terms in molecular biology may be found, for example, in Benjamin Lewin, Genes VII, published by Oxford University Press, 2000 (ISBN 019879276X); Kendrew et al. (eds.); The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341); and other similar technical references.

As used herein, “a” or “an” may mean one or more. As used herein when used in conjunction with the word “comprising,” the words “a” or “an” may mean one or more than one. As used herein “another” may mean at least a second or more. Furthermore, unless otherwise required by context, singular terms include pluralities and plural terms include the singular.

As used herein, “about” refers to a numeric value, including, for example, whole numbers, fractions, and percentages, whether or not explicitly indicated. The term “about” generally refers to a range of numerical values (e.g., +/−5-10% of the recited value) that one of ordinary skill in the art would consider equivalent to the recited value (e.g., having the same function or result). In some instances, the term “about” may include numerical values that are rounded to the nearest significant figure.

II. The Present Invention

Delivery of therapeutics to the GI tract is complicated by such factors as difficulty in targeting the proper region of the GI tract, variable pH profiles present in the different regions, and the enzymatic environment of the GI tract. Such complications can be increased when attempting to deliver drugs to patients whose disease or condition results in fundamental changes to the GI tract. For example, most patients suffering from Inflammatory Bowel Disease (IBD) suffer from diarrhea and therefore the GI transit time shortens dramatically from that of healthy subjects. Thus, the development of delivery systems that can be tuned to release therapeutic agents in selected areas of the GI tract would be very helpful in improving treatment and prevention of diseases and conditions localized to the GI tract.

The present invention is directed to a delivery system that allows for targeted release of therapeutics in selected regions of the GI tract, including the small intestine and/or large intestine/colon. For example, and in certain aspects, the delivery system is adapted to deliver a certain type of therapeutic, such as a protein-based therapeutic, to a certain area of the GI tract, such the small intestine or colon of a subject.

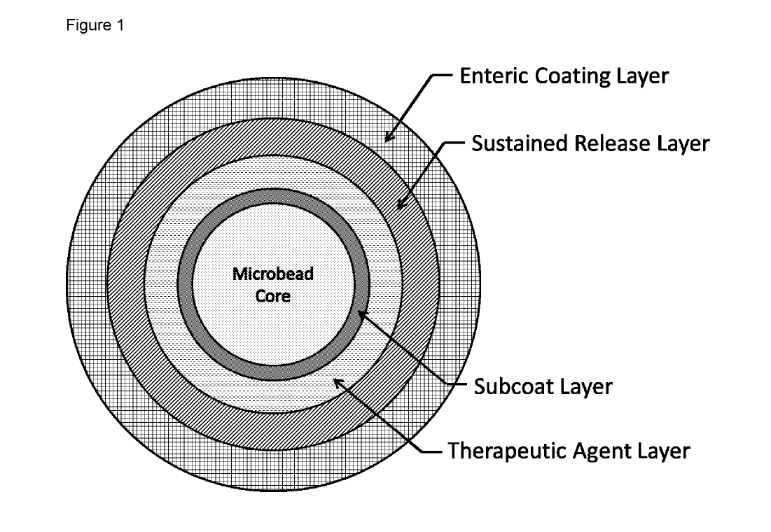

The present invention can be generally defined as a vehicle for delivery of a therapeutic agent to the GI tract, where the delivery vehicle (DV) comprises a microbead coated with layers that include a subcoat layer and a therapeutic agent layer. The delivery vehicles of the invention will also often include an enteric coating layer and/or a sustained release layer. In each of the embodiments of the delivery vehicles of the invention, the microbead is solid and comprises a one or more physiologically inert substances. The subcoat layer substantially coats the microbead and generally comprises a polymer, which increases coating efficiency by the therapeutic agent layer. The therapeutic agent layer substantially coats the subcoat layer and generally comprises a therapeutic agent and one or more excipients. When present, the enteric coating layer substantially coats the therapeutic agent layer or sustained release layer and generally comprises a pH-resistant composition. When present, the sustained release layer substantially coats the therapeutic agent layer (i.e., it is between the therapeutic agent layer and the enteric coating layer). FIG. 1 provides the cross-section of a delivery vehicle of the invention that includes each of a microbead, a subcoat layer, a therapeutic agent layer, a sustained release layer, and an enteric coating layer.

In one embodiment, the invention is directed to delivery vehicles. In a first aspect the DV comprises a microbead coated with a subcoat layer and a therapeutic agent layer. In a second aspect the DV comprises a microbead coated with a subcoat layer, a therapeutic agent layer, and a sustained release layer. In a third aspect the DV comprises a microbead coated with a subcoat layer, a therapeutic agent layer, and an enteric coating layer. In a fourth aspect the DV comprises a microbead coated with a subcoat layer, a therapeutic agent layer, a sustained release layer, and an enteric coating layer.

In a further embodiment, the invention is directed to a DV that delivers a therapeutic agent to the GI tract of a subject. In a first aspect, the DV comprises a microbead coated with a subcoat layer and a therapeutic agent layer. In a second aspect, the DV comprises a microbead coated with a subcoat layer, a therapeutic agent layer, and a sustained release layer. In a third aspect, the DV comprises a microbead coated with a subcoat layer, a therapeutic agent layer, and an enteric coating layer. In a fourth aspect, the DV comprises a microbead coated with a subcoat layer, a therapeutic agent layer, a sustained release layer, and an enteric coating layer. In selected aspects, these DVs deliver a therapeutic agent to a subject, for example to the small intestine of a subject.

In another embodiment, the invention is directed to a colonic DV that delivers a therapeutic agent to the colon of a subject. In a first aspect, the colonic DV comprises a microbead coated with a subcoat layer and a therapeutic agent layer. In a second aspect, the colonic DV comprises a microbead coated with a subcoat layer, a therapeutic agent layer, and a sustained release layer. In a third aspect, the colonic DV comprises a microbead coated with a subcoat layer, a therapeutic agent layer, and an enteric coating layer. In a fourth aspect, the colonic DV comprises a microbead coated with a subcoat layer, a therapeutic agent layer, a sustained release layer, and an enteric coating layer.

### Microbeads

The microbeads that may be used in each of the delivery vehicles of the present invention are generally comprised of one or more physiologically inert substances. Because the microbeads are simply the carrier upon which the therapeutic agent is layered, the substances which comprise the microbeads need only be characterized by being compactable (i.e., able to form a microbead and maintain such a form during manufacture, storage, and administration of the DV, and transit of the DV to the GI tract of a subject) as well as non-toxic and non-immunogenic to the subject to which the DVs are administered.

The microbeads may be comprised of one or more physiologically inert substances that include, but are not limited to, sugars, starches, microcrystalline cellulose (MCC), biodegradable polymers (e.g., PLGA, a copolymer of L-lactic acid and glycolic acid), and sodium or calcium phosphates, and mixtures thereof. Exemplary sugars include, but are not limited to, one or more of lactose, sucrose, mannitol, trehalose, maltodextrin, dextrose, fructose and polysaccharide. Exemplary starches include, but are not limited to, one or more of corn starch, pea starch, potato starch, rice starch, tapioca starch, wheat starch, modified starch, and pre-gelatinized starch.

While the microbead will generally be round in shape, it will be understood that other shapes, including oval shapes (e.g. egg shaped), square shapes (e.g. cubed), diamonds, pyramids, and rectangular shapes are also acceptable, and as well as amorphous shapes. The microbeads that may be used in the DVs of the invention will generally have an average particle diameter of between about 1 and 1000 μm. Suitable ranges of particles sizes include, but are not limited to, 1-1000 μm, 1-900 μm, 1-800 μm, 1-700 μm, 1-600 μm, 1-500 μm, 1-400 μm, 1-300 μm, 1-200 μm, 1-100 μm, 100-1000 μm, 100-900 μm, 100-800 μm, 100-700 μm, 100-600 μm, 100-500 μm, 100-400 μm, 100-300 μm, 100-200 μm, 200-1000 μm, 200-900 μm, 200-800 μm, 200-700 μm, 200-600 μm, 200-500 μm, 200-400 μm, 200-300 μm, 300-1000 μm, 300-900 μm, 300-800 μm, 300-700 μm, 300-600 μm, 300-500 μm, 300-400 μm, 400-1000 μm, 400-900 μm, 400-800 μm, 400-700 μm, 400-600 μm, 400-500 μm, 500-1000 μm, 500-900 μm, 500-800 μm, 500-700 μm, 500-600 μm, 600-1000 μm, 600-900 μm, 600-800 μm, 600-700 μm, 700-1000 μm, 700-900 μm, 700-800 μm, 800-1000 μm, 800-900 μm, and 900-1000 μm.

Commercially-available microbeads may be used, or the microbeads may be made de novo. Suitable commercially-available microbeads include, but are not limited to, D-mannitol microbeads (Nonpareil-108®; grade: 32-42; particle size: 355-500 μm; Freund Corporation, Tokyo, Japan), sugar spheres (SUGLETS® Sugar Spheres, grade 45/60; size: 250-355 um; Colorcon Inc. Pa., USA), microcrystalline cellulose spheres (Celphere™; grade: cp-305; particle size: 300-500 um; AsahiKASEI Chemical Corp. Tokyo, Japan).

In some aspects of the invention the microbeads may also comprise a medicament in addition to the physiologically inert substances, or in place of the physiologically inert substances. Such DVs can thus comprise two active agents, the therapeutic agent of the therapeutic agent layer and the medicament of the microbeads. While the therapeutic agent of the therapeutic agent layer is a protein, as discussed below, the medicament of the microbeads can be a non-protein medicament. Suitable medicaments include, but are not limited to, proto-peptides, nucleic acids, gene constructs, analgesics, antibiotics, anti-viral agents, and anti-cancer drugs, for example.

### Subcoat Layer

To prevent uncontrolled attrition of the microbeads, whether during manufacture, storage, delivery, or transit of the DV, a subcoat layer is provided on the surface of the microbeads. The subcoat layer can also help to prevent aggregation of the microbeads during production of the DV. The subcoat layer can further serve to increase coating efficiency by the therapeutic agent layer.

Suitable components of the subcoat layer include, but are not limited to, one or more of ammonium alginate, cellaburate, chitosan, colophony, copovidone, ethylene glycol and vinyl alcohol grafted copolymer, gelatin, hydroxypropyl cellulose, hypromellose, hypromellose acetate succinate, polymethacrylate, poly(methyl vinyl ether/maleic anhydride), polyvinyl acetate dispersion, polyvinyl acetate phthalate, polyvinyl alcohol, polyvinylpyrrolidone (PVP), povidone, pullulan, pyroxylin, and shellac.

In preferred embodiments, the subcoat layer will completely cover the surface of the microbead. However, embodiments where the subcoat layer only substantially coats the microbeads are also acceptable. As used herein, “substantially coats” means that a layer covers at least 80% of the surface of the microbead or an underlying layer previously applied to the microbead. In preferred embodiments, the layer substantially coats at least 81%, 82%, 83%, 84%, 85%, 86%, 87%, 88%, 89%, 90%, 91%, 92%, 93%, 94%, 95%, 96%, 97%, 98%, or 99% of the surface of the microbead or the underlying layer previously applied to the microbead.

The amount of subcoat layer added to the microbeads can be conveniently defined based on the amount of weight that is imparted by the subcoat layer to the microbeads. In general, the amount of weight gain imparted by the subcoat layer to the microbead ranges between about 0.1 and 10%. Suitable ranges of weight gain include, but are not limited to, 0.1-9%, 0.1-8%, 0.1-7%, 0.1-6%, 0.1-5%, 0.1-4%, 0.1-3%, 0.1-2%, 0.1-1%, 0.5-9%, 0.5-8%, 0.5-7%, 0.5-6%, 0.5-5%, 0.5-4%, 0.5-3%, 0.5-2%, 0.5-1%, 1-9%, 1-8%, 1-7%, 1-6%, 1-5%, 1-4%, 1-3%, 1-2%, 2-9%, 2-8%, 2-7%, 2-6%, 2-5%, 2-4%, 2-3%, 3-9%, 3-8%, 3-7%, 3-6%, 3-5%, and 3-4%.

The subcoat layer may be applied to the microbead using means that include, but are not limited to, spray coating, dip coating, powder coating, hot melt-extrusion, and spray drying.

In a preferred aspect of the invention, the microbeads are coated with a solution of 2.5% polyvinylpyrrolidone (PVP; Kollidon® 30) to achieve a PVP subcoat layer that imparts a 1% weight gain to the microbeads.

### Therapeutic Agent Layer

The therapeutic agent layer that is included in the DVs of the present invention comprises a therapeutic agent and one or more excipients.

Suitable therapeutic agents that may be used include, but are not limited to one or more of an antibody, an antiviral, an antifungal, an antibiotic, an anticancer agent, an analgesic, an anticoagulant, an antidepressant, an antiepileptic, an antipsychotic, and a sedative. In preferred embodiments of the invention, the therapeutic agent is a protein-based therapeutic agent. Suitable protein-based therapeutic agents include, but are not limited to, antibodies, fragments of antibodies and fusion constructs thereof (including single-chain variable fragment (scFv) antibodies and hybrid antibodies), antibody-drug conjugates, pegylated antibodies, polypeptides, proteins, and peptides (such as antimicrobial peptides). In a particular embodiment of the invention, the therapeutic agent is an antibody having binding specificity for C. difficile toxin A and/or toxin B, e.g., as described in international patent publication WO 2016/127104, herein incorporated by reference in its entirety.

The one or more excipients that may be included in the therapeutic agent layer may be any pharmaceutically acceptable excipient that can be used in conjunction with the therapeutic agent. The excipient is generally one that stabilizes the therapeutic agent, whether in the context of the DV or upon release of the therapeutic agent from the DV, or both. The excipient may additionally be one that aids in attachment of the therapeutic agent to the microbeads in the form of a layer. Suitable excipients include, but are not limited to, one or more of polyvinylpyrrolidone (PVP), polyethylene glycol (PEG), polyvinyl acetate (PVA), hydroxypropyl cellulose (HPC), sucrose, trelose, acacia, tragacanth, gelatin, starch, pregelatinized starch, alginic acid, cellulose, methyl cellulose, ethyl cellulose, sodium carboxy methyl cellulose, polymethacrylate, asparagine, dextran, glycine, inulin, lactose, anhydrous lactose, monohydrate, mannitol, raffinose, and trehalose.

In preferred embodiments, the therapeutic agent layer will completely cover the surface of the layer below it. However, embodiments where the therapeutic agent layer only substantially coats the layer below it are also acceptable, where the term “substantially coats” is as defined herein.

The amount of therapeutic agent layer added to the microbeads can be conveniently defined based on the amount of weight that is imparted by the therapeutic agent layer to the microbeads (which include any layers previous applied to the beads). The weight imparted by the therapeutic agent layer, and the corresponding thickness of the layer, can be controlled, thereby allowing DVs with different amounts of therapeutic agents coated on the microbeads to be prepared. The ability to vary the amount of therapeutic agent on the beads allows the delivery vehicles to be customized for selected diseases and conditions, and even customized for a particular subject having a unique set of characteristics and symptoms.

In general, the amount of weight gain imparted by the therapeutic agent layer to the microbeads ranges from between about 0.1 and 400%. Suitable ranges of weight gain also include, but are not limited to, 0.1-30%, 0.1-25%, 0.1-20%, 0.1-15%, 0.1-10%, 0.1-5%, 1-30%, 1-25%, 1-20%, 1-15%, 1-10%, 1-5%, 5-30%, 5-25%, 5-20%, 5-15%, 5-10%, 10-30%, 10-25%, 10-20%, 10-15%, 15-30%, 15-25%, 15-20%, 20-30%, 20-25%, 25-30%, 1-375%, 5-350%, 10-325%, 15-300%, 20-275%, 25-250%, 30-225%, 35-200%, 40-175%, 45-150%, 50-150%, 55-125%, 55-100%, 50-350%, 100-300%, 150-250%, 50-300%, 50-200%, and 50-100%.

The therapeutic agent layer may be applied to the microbead using means that include, but are not limited to, spray coating, dip coating, powder coating, hot melt-extrusion, and spray drying.

### Enteric Coating Layer

To promote release of the therapeutic agent from the DVs in a selected area of the GI tract, such as the colon, an enteric coating layer may be applied to the therapeutic agent layer, or the sustained release layer if such a layer is present.

When present, the enteric coating layer comprises a pH-resistant composition whereby the enteric coating layer only dissolves when the DVs are present in an environment having a pH of 5.0 or more. When the DVs are targeted to the colon, the enteric coating layer will be one that only dissolves when the colonic DVs are present in an environment having a pH of 7.0 or more. In certain embodiments of the invention, the enteric coating layer comprises a pH-resistant composition whereby the enteric coating layer only dissolves when the colonic DVs are present in an environment having a pH of more than 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, or 8.5.

Suitable components of the enteric coating layer include, but are not limited to, one or more of inulin, shellac, methacrylated inulin, pectin, chitosan, Eudragit® FS 30D, Eudragit® S 100 and Eudragit® S12,5. Depending on the components included in the enteric coating layer, a plasticizer may be included as a component of the enteric coating layer. Suitable plasticizers include, but are not limited to, PlasACRYL™ T20, acetyltributyl citrate, acetyltriethyl citrate, alpha tocopherol, benzyl benzoate, butyl stearate, chlorobutanol, dibutyl phthalate, dibutyl sebacate, diethyl phthalate, dimethyl phthalate, dipropylene glycol, glycerin, mannitol, petrolatum and lanolin alcohols, polyethylene glycol, polyoxyethylene sorbitan and fatty acid esters, propylene glycol, pyrrolidone, sorbitol, triacetin, tributyl citrate, triethyl citrate, and tricaprylin.

In preferred embodiments, the enteric coating layer will completely cover the surface of the layer below it. However, embodiments where the enteric coating layer only substantially coats the layer below it are also acceptable, where the term “substantially coats” is as defined herein.

The amount of enteric coating layer added to the microbeads can be conveniently defined based on the amount of weight that is imparted by the enteric coating layer to the microbeads (which include any layers previously applied to the beads). The weight imparted by the enteric coating layer, and the corresponding thickness of the layer, can be controlled, thereby allowing DVs with different amounts of enteric coating to be applied to the microbeads. The ability to vary the amount of enteric coating on the beads allows the delivery vehicles to be customized for release of the therapeutic agent in selected environments (e.g., a certain pH) and/or in selected regions of the GI tract. The delivery vehicles can thus be customized for selected diseases and conditions, and even customized for a particular subject having a unique set of characteristics and symptoms.

In general, the amount of weight gain imparted by the enteric coating layer to the microbeads ranges from between about 5 and 50%. Suitable ranges of weight gain also include, but are not limited to, 5-40%, 5-35%, 5-30%, 5-25%, 5-20%, 5-15%, 5-10%, 10-40%, 10-35%, 10-30%, 10-25%, 10-20%, 10-15%, 15-40%, 15-35%, 15-30%, 15-25%, 15-20%, 20-40%, 20-35%, 20-30%, 20-25%, 25-40%, 25-35%, 25-30%, 30-40%, and 30-35%.

The enteric coating layer may be applied to the microbead using means that include, but are not limited to, spray coating, dip coating, powder coating, hot melt-extrusion, and spray drying.

### Sustained Release Layer

To provide additional flexibility to DVs in terms of the location, manner, and speed of therapeutic agent release from the DVs, a sustained release layer may be included in the DVs.

When present, the sustained release layer comprises one or more components that slow or delay the release of the therapeutic agent from the surface of the microbeads. Suitable components include, but are not limited to, one or more of acacia, agar, alginic acid, aliphatic polyester, calcium alginate, carbomer, carrageenan, cellaburate, cellulose acetate, ceratonia, copovidone, gellan gum, guar gum, hydroxyethylmethyl cellulose, hydroxypropyl betadex, hydroxypropyl cellulose, hypromellose, methylcellulose, polycarbophil, poly(DL-lactic acid), polymethacrylate, polyoxylglyceride, polyvinyl acetate dispersion, shellac, sodium alginate, starch modified, xanthan gum, zein, Eudragit® RL, Eudragit® RL 30D, Eudragit® RL PO, Eudragit® RL 100, Eudragit® RL 12,5, Eudragit® RS 30 D, Eudragit® RS PO, Eudragit® RS 100, Eudragit® RS 12,5, Eudragit® NE 30 D, Eudragit® NE 40 D, and Eudragit® NM 30 D.

In preferred embodiments, the sustained release layer will completely cover the surface of the layer below it. However, embodiments where the sustained release layer only substantially coats the layer below it are also acceptable, where the term “substantially coats” is as defined herein.

The amount of sustained release layer added to the microbeads can be conveniently defined based on the amount of weight that is imparted by the sustained release layer to the microbeads (which include any layers previous applied to the beads). The weight imparted by the sustained release layer, and the corresponding thickness of the layer, can be controlled, thereby allowing DVs with different amounts of sustained release layers to be applied to the microbeads. The ability to vary the amount of the sustained release layer on the beads allows the delivery vehicles to be customized for release of the therapeutic agent over selected periods of time and rates of release. The delivery vehicles can thus be customized for selected diseases and conditions, and even customized for a particular subject having a unique set of characteristics and symptoms.

In general, the amount of weight gain imparted by the sustained release layer to the microbeads ranges from between about 1 and 50%. 5-40%, 5-35%, 5-30%, 5-25%, 5-20%, 5-15%, 5-10%, 10-40%, 10-35%, 10-30%, 10-25%, 10-20%, 10-15%, 15-40%, 15-35%, 15-30%, 15-25%, 15-20%, 20-40%, 20-35%, 20-30%, 20-25%, 25-40%, 25-35%, 25-30%, 30-40%, and 30-35%.

The sustained release layer may be applied to the microbead using means that include, but are not limited to, spray coating, dip coating, powder coating, hot melt-extrusion, and spray drying.

### Means for Preparing the Delivery Vehicles

The present invention is also directed to means for preparing the DVs of the present invention.

In general, the DVs of the present invention are prepared by successively coating microbeads with the different layers defined herein. For example, the invention comprises a method whereby in step (a) a subcoat layer is applied to a microbead and the subcoat layer substantially coats the microbead. In step (b) a therapeutic agent layer is applied to the microbead produced in (a) and the therapeutic agent layer substantially coats the subcoat layer. When the DVs include a sustained release layer, step (c) is performed whereby the sustained release layer is applied to the microbead produced in (b) and the sustained release layer substantially coats the microbead produced in (b). When the DVs include an enteric coating layer, the enteric coating layer is applied to the microbead produced in (b) or (c) and the enteric coating layer substantially coats the microbead produced in (b) or (c).

The different layers are applied to the microbeads using means that include, but are not limited to, spray coating, dip coating, powder coating, hot melt-extrusion, and spray drying.

Spray coating is a technique used to produce coated microbeads by spraying a liquid onto the surface of fluidized microbeads supported by air or a gas. The microbeads are continuously fluidized in the fluid bed chamber so that the coating of therapeutic agents or excipients is uniform. This is the preferred method for coating many thermally-sensitive materials such as foods and pharmaceuticals. Air is the heated drying medium; however, if the liquid is a flammable solvent such as ethanol or the product is oxygen-sensitive then nitrogen may be used. This technique is particularly beneficial when coating microbeads with a protein-based therapeutic agent, such as an antibody, as it minimizes shear and heat stresses that could lead to structure perturbations, protein aggregation or loss of activity. The actual temperature of a product surface is usually much lower than the inlet air temperature due to an evaporative cooling effect. When dealing with an extreme fragile or heat-sensitive protein, the strategy of Design of Experiments (DoE) can be used to tune the process temperature, atomization temperature, air flow rate, etc. to optimize the process parameters. For example, inlet air temperature is preferably lower than the onset unfolding temperature of proteins; however, higher inlet air temperature can promote the process of spray coating, saving time and labor. In this case, the optimal combination of process parameters may be determined using a DoE strategy.

The particular characteristics of a spray coating methodology practiced in the production of the DVs of the invention, such as nozzle size, bowel volume, inlet temperature, product temperature, atomization pressure, microclimate air pressure, and feed rate will vary based on such factors as the identity and size of the microbead; the identity of the subcoat layer, therapeutic agent layer, sustained release layer, and enteric coating layer; and the amount of subcoat layer, therapeutic agent layer, sustained release layer, and enteric coating layer added to the microbead, among others.

### Small Intestine and Colon

As used herein, the “small intestine” includes the duodenum, jejunum and ileum. The duodenum is about 20-25 cm in length and it starts at the stomach and includes pancreas ducts. The jejunum is about 2.5 m in length and it is the middle portion of the small intestine, connecting the duodenum to the ileum. The ileum is about 3 m in length and it joins to the cecum of the large intestine at the ileocecal junction.

As used herein, the “colon” is synonymous with the large intestine and includes the cecum, rectum, and anal canal. It also includes the appendix, which is attached to the cecum. The colon can be divided into Cecum (first portion of the colon) and appendix, Ascending colon (ascending in the back wall of the abdomen), Right colic flexure (flexed portion of the ascending and transverse colon apparent to the liver), Transverse colon (passing below the diaphragm), Left colic flexure (flexed portion of the transverse and descending colon apparent to the spleen, Descending colon (descending down the left side of the abdomen), Sigmoid colon (a loop of the colon closest to the rectum), Rectum and Anus.

### Methods of Treatment and Prevention

The delivery vehicles of the invention can be used in methods of treating or preventing a disease or condition in a subject. These methods generally comprise administering a therapeutically-effective amount of one or more of the delivery vehicles as defined herein to a subject in need thereof, thereby treating the disease or condition in the subject.

Suitable diseases and conditions include, but are not limited to, intestinal inflammatory diseases, autoimmune diseases, inflammatory bowel disease (IBD), celiac disease, irritable bowel syndromes, bacterial infections, viral infections, fungal infections, Clostridium difficile infection (CDI), and gastric intestinal tract malignancies (including, but not limited to, esophageal cancer, stomach cancer, and colorectal cancer).

When the disease or condition is IBD, the therapeutic agent may be one or more of an antibody, cytokine or therapeutic protein that blocks the activity of secreted immune defense molecules, blocks bacterial or viral virulence, regulates immune function, or regulates enterocyte function.

When the disease or conditions is CDI, the therapeutic agent may be an antibody or fragment thereof that blocks the activity C. difficile enterotoxin TcdA or Tcd B, blocks the activity of both TcdA and Tcd B, or blocks the activity of other virulence factors, bacterial proliferation or survival. For example, the therapeutic agent can be an antibody against C. difficile TcdA and/or TcdB for the treatment of C. difficile infection as described in international patent publication WO 2016/127104, which is herein incorporated by reference in its entirety. Exemplary therapeutic agents include, but are not limited to, BSA, rabbit IgG, human IgG, chimeric ABAB-IgG1, and humanized ABAB-IgG1.

In a particular aspect, the invention is direct to a method of treating or preventing a disease symptom induced by C. difficile in a subject comprising administering a therapeutically-effective amount of a delivery vehicle of the invention to a subject having C. difficile infection or a risk of developing C. difficile infection.

In another particular aspect, the invention is directed to a method of neutralizing C. difficile toxin TcdA and/or TcdB in a subject infected by C. difficile comprising administering a therapeutically-effective amount of a delivery vehicle of the invention to a subject having C. difficile infection.

In further particular aspect, the invention is direct to a method of treating or preventing C. difficile infection in a subject comprising administering a therapeutically-effective amount of a delivery vehicle of the invention to a subject having C. difficile infection or a risk of developing C. difficile infection. These same methods can be used to treat CDI, as defined herein.

The delivery vehicles can also be used in immunoprophylaxis in order to prevent immediate CDI threats. In addition, passive immunoprophylaxis can be used to prevent both immediate and longer-term CDI threats. Each approach has its own particular advantages and is suitable to target a particular high-risk population. These methods generally comprises administering a therapeutically-effective amount of one or more of the delivery vehicles as defined herein to a subject a risk of developing C. difficile infection.

As used herein, the terms “treat”, “treating”, and “treatment” have their ordinary and customary meanings, and include one or more of: blocking, ameliorating, or decreasing in severity and/or frequency a symptom of a disease or condition in a subject. Treatment means blocking, ameliorating, decreasing, or inhibiting by about 1% to about 100% versus a subject in which the methods of the present invention have not been practiced. Preferably, the blocking, ameliorating, decreasing, or inhibiting is about 100%, 99%, 98%, 97%, 96%, 95%, 90%, 80%, 70%, 60%, 50%, 40%, 30%, 20%, 10%, 5% or 1% versus a subject in which the methods of the present invention have not been practiced.

As used herein, the terms “prevent”, “preventing” and “prevention” have their ordinary and customary meanings, and include one or more of, stopping, averting, avoiding, or blocking a disease or condition in a subject. Prevention means stopping by at least about 95% versus a subject to which the prevention has not been administered. Preferably, the stopping is about 100%, about 99%, about 98%, about 97%, about 96% or about 95%. The results of the prevention may continue for a period of days (such as 1, 2, 3, 4, 5, 6 or 7 days), weeks (such as 1, 2, 3 or 4 weeks) or months (such as 1, 2, 3, 4, 5, 6 or more months).

The method of treating and preventing provided herein can be supplemented by also administering a therapeutically-effective amount of an antibiotic to the subject. Preferably, the antibiotic will have antibacterial activity against C. difficile. The antibiotic may be administered separately or concurrently with the DVs of the invention. The antibiotic may also be present in the microbeads of the DVs.

### Formulations and Doses

While the DVs of the invention may be administered directly to a subject, the methods of the present invention may also be based on the administration of a pharmaceutical formulation comprising (i) one or more populations of a delivery vehicle of the invention and (ii) a pharmaceutically acceptable carrier or diluent. Thus, the invention includes pharmaceutical formulations comprising one or more populations of the delivery vehicles defined herein and a pharmaceutically acceptable carrier or diluent.

Pharmaceutically acceptable carriers and diluents are commonly known and will vary depending on the particular mode of administration. Examples of generally used carriers and diluents include, without limitation: saline, buffered saline, dextrose, water-for-injection, glycerol, ethanol, and combinations thereof, stabilizing agents, solubilizing agents and surfactants, buffers and preservatives, tonicity agents, bulking agents, and lubricating agents.

The delivery vehicles and pharmaceutical formulations comprising the delivery vehicles may be administered to a subject using modes and techniques known to the skilled artisan. Acceptable modes of delivery include, but are not limited to, oral, anal, via intravenous injection or aerosol administration. Other modes include, without limitation, intradermal, subcutaneous (s.c., s.q., sub-Q, Hypo), intramuscular (i.m.), intraperitoneal (i.p.), intra-arterial, intramedulary, intracardiac, intra-articular (joint), intrasynovial (joint fluid area), intracranial, intraspinal, and intrathecal (spinal fluids).

Depending on the means of administration, the dosage may be administered all at once, such as with an oral formulation in a capsule or liquid, or slowly over a period of time, such as with an intramuscular or intravenous administration.

The amount of delivery vehicle, alone or in a pharmaceutical formulation, administered to a subject is an amount effective for the treatment or prevention of a particular disease or condition. Thus, therapeutically-effective amounts are administered to subjects when the methods of the present invention are practiced. In general, between about 1 ug/kg and about 1000 mg/kg of a particular delivery vehicle per body weight of the subject is administered. Suitable ranges also include between about 50 ug/kg and about 500 mg/kg, and between about 100 ug/kg and about 100 mg/kg. However, the amount of delivery vehicle administered to a subject will vary between wide limits, depending upon the location, source, extent and severity of the disease or condition, the identity of the therapeutic agent, the age and condition of the subject to be treated, etc. A physician will ultimately determine appropriate dosages to be used.

Administration frequencies of the delivery vehicles and pharmaceutical formulations comprising the delivery vehicles will vary depending on factors that include the identity and location of the disease or condition, the identity of the therapeutic agent, and the mode of administration. Each formulation may be independently administered 4, 3, 2 or once daily, every other day, every third day, every fourth day, every fifth day, every sixth day, once weekly, every eight days, every nine days, every ten days, bi-weekly, monthly and bi-monthly.

The duration of treatment or prevention will be based on identity, location and severity of the disease or condition being treated and will be best determined by the attending physician. However, continuation of treatment is contemplated to last for a number of days, weeks, or months.

In each embodiment and aspect of the invention, the subject is a human, a non-human primate, bird, horse, cow, goat, sheep, a companion animal, such as a dog, cat or rodent, or other mammal.

The invention also provides a kit comprising one or more containers filled with one or more populations of the delivery vehicles of the invention or pharmaceutical formulations comprising the delivery vehicles. The kit may also include instructions for use. Associated with the kit may further be a notice in the form prescribed by a governmental agency regulating the manufacture, use or sale of pharmaceuticals or biological products, which notice reflects approval by the agency of manufacture, use or sale for human administration.

### III. EXAMPLES

### Example 1—Preparation of BSA Coated Microbeads

In a first group of experiments, BSA (bovine serum albumin) was used as a model therapeutic agent for the development of the colonic delivery vehicles of the invention because the structure and degradation under stress profile of the protein has been studied extensively and it is well understood [9-16].

BSA consists of 583 amino acids, with a molecular weight of 66.5 kDa [17]. It contains 17 disulfide bridges in the tertiary structure, which render BSA with a high propensity to aggregate, once the protein unfolds upon external stress and exposes the disulfides to solvents [18]. Thus, BSA is a good protein in which to monitor protein degradation and aggregation during the multiple processes.

As discussed below, BSA was first spray layered onto the surface of D-mannitol microbeads (to form a therapeutic agent layer), after which the aggregation profile and the secondary/tertiary structures of BSA were examined. The BSA layered beads were also challenged in an accelerated stability study. Secondly, the BSA layered beads were coated with an enteric polymer EUDRAGIT® FS 30 D to different thicknesses (to form the enteric coating layer). The in vitro release of final products were examined and showed colonic targeting release.

### Materials

D-mannitol beads (32-42 mesh) were purchased from Freund Corporation (Shinjuku-ku, Japan). Bovine serum albumin was obtained from Sigma Aldrich (St. Louis, Mo.). Polyvinylpyrrolidone (PVP; Kollidon® 30) was a gift from BASF (Tarrytown, N.Y.). EUDRAGIT® FS 30 D (Poly(methyl acrylate-co-methyl methacrylate-co-methacrylic acid) 7:3:1) was a gift from Evonik (Parsippany, N.J.). HPLC grade water was purchased from Sigma Aldrich (St. Louis, USA).

Spray coating of BSA

Spray coating was performed in Bosch Solidlab 1 fluid bed system with a nozzle size of 0.6 mm and a bowel volume of 0.3 L. The atomization pressure was 0.6 bar and the microclimate air pressure was 0.3 bar. The inlet air temperature was 45° C. while the product temperature was 35° C., which was below the onset unfolding temperature of BSA. To prevent bead attrition, a solution of 2.5% PVP (Kollidon® 30) was first prepared and sprayed at 0.74 g/min to D-mannitol beads to achieve a subcoat layer of 1% weight gain. The feed rate was 0.74 to 1 g/min. To apply the BSA layer, the spray solution was prepared as 100 mg/ml of BSA and 2% (w/v) of PVP in water. The total time of spraying was 2 h. 5 grams of BSA in total were spray coated onto the surface of 50 grams of the pre-coated beads.

The BSA layered beads were reconstituted in HPLC grade water followed by filtration. The filtrate was measured for protein concentration using a DU 800 UV/Vis Spectrophotometer (Brea, Calif.) at a wavelength of 280 nm and an absorptivity A^{280 nm}_{1 g/L} of 0.667 (from Certificate of Analysis of BSA). The loading efficiency was calculated using the following equation: Loading efficiency=Concentration of filtrate * Volume/Weight of BSA sprayed. The yield of spray layered BSA was 89% calculated from total mass recovery, which is much higher than the typical recovery rate in spray drying [20, 21]. There was very few agglomerates but lots of fines sticking to the walls or filters of the fluid bed, which explains the comparatively low yield.

Characterization of BSA layered beads

Insoluble aggregates—BSA layered beads were reconstituted in HPLC grade water. The protein concentration of the solution was measured using a DU 800 UV/Vis Spectrophotometer (Brea, Calif.) at a wavelength of 280 nm and an absorptivity A^{280 nm}_{1 g/L} of 0.667. The solution was then filtered using a Millex®-GP 0.22 μm PES filter (Tullagreeen, Ireland), after which the protein concentration was measured again. The difference of protein concentration before and after filtration (0.22 um) was used to quantitate the insoluble aggregates of BSA.

The number of insoluble BSA aggregates did not change significantly after spray coating (student t test, n=3, P<0.05), with mean=0.83%, SD=0.15% in commercial BSA, and mean=0.69%, SD=0.07% in reconstituted BSA samples. The limitation of this method was that insoluble aggregates smaller than 0.22 μm may also have passed through the filter since the reported hydrodynamic size of BSA monomer was 7 nm.

Soluble aggregate determination—The filtrate of reconstituted protein solution after filtration was diluted to 0.5 mg/mL and analyzed for soluble aggregates using size exclusion chromatography. A Superose 12 10/300 GL column (Pittsburgh, GE Healthcare) was coupled with AKTA FPLC system. A UV detector was used to quantitate the protein concentration at 280 nm. The chromatograph was exported to a data analysis software OriginPro 9.0 (OriginLab Corporation, Northampton, Mass.) and fitted into a combination of monomer, dimer and trimer of BSA.

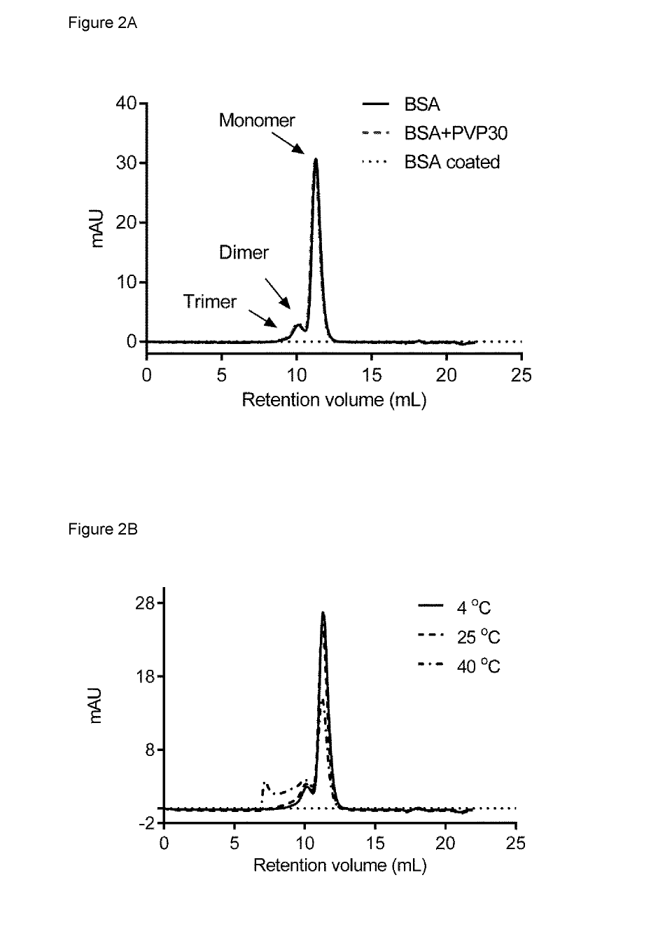

For soluble aggregates, the size exclusion chromatograph of native BSA freshly reconstituted showed a major monomer peak around 11.2 mL (elution volume), overlapped with a dimer peak at 10.1 mL and a trimer peak at 9.4 mL (FIG. 2A); these elution volumes were based on the calibration of the SEC column with molecular weight markers. The monomer, dimer and trimer content of BSA did not change after mixing with the binder PVP or after spray coating, which indicates there was no significant change in the extent of soluble aggregate formation. After storage of the BSA beads under 25° C. and 40° C. for 38 days, the monomer percentage decreased from 86.7% to 76.4% and 50.83%, respectively, while the high molecular weight species including dimer and trimers increased from 13.3% to 23.6% and 49.2%, respectively (FIG. 2B). No fragmentation of BSA was seen in any samples. This data correlated with the insoluble aggregates increase seen after storage at 40° C. Soluble aggregates may form insoluble aggregates given enough time under stressed conditions.

Secondary structure determination—The secondary structure of protein was measured by a circular dichroism (CD) spectrometer JASCO J-810 (Easton, USA). The CD spectrum was collected from 190 nm to 260 nm in a 0.05 mm cuvette. The data pitch was 1 nm and the bandwidth was 1 nm. Three accumulations were averaged out. The CD spectrum was analyzed for secondary structure components using DichroWeb. The algorithm SELCON 3 with reference set 4 was used for analysis.

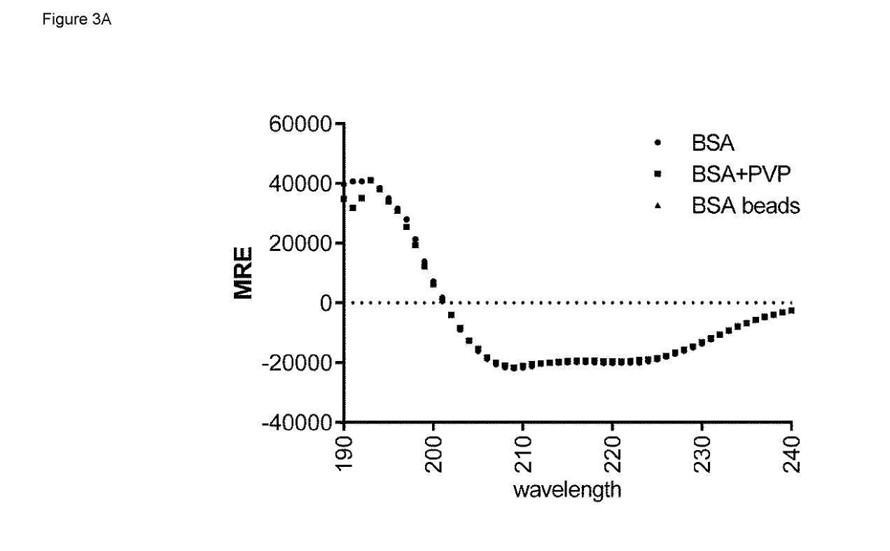

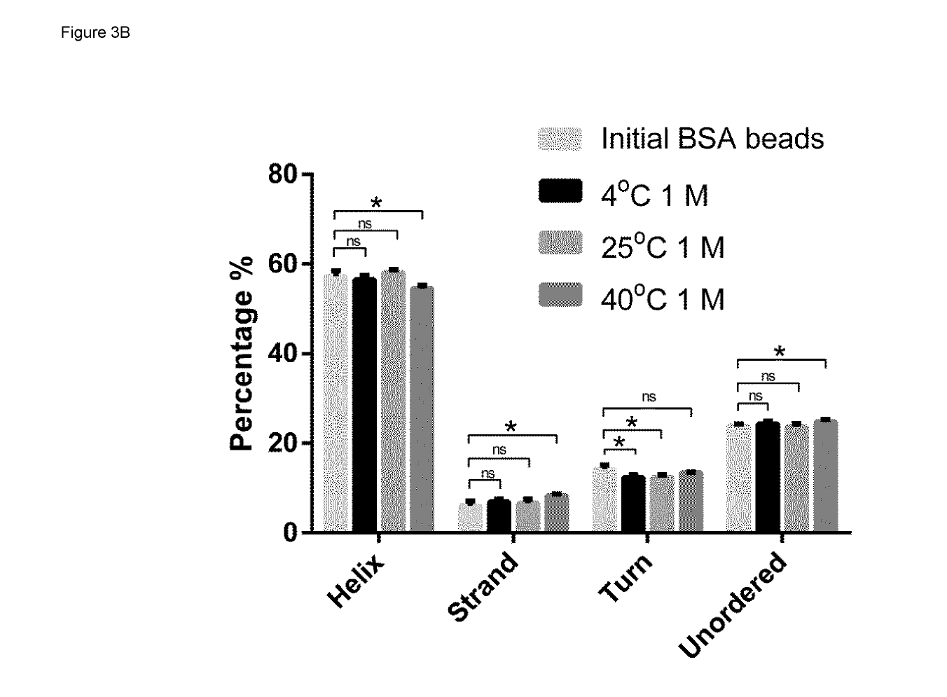

The far UV-CD spectrum of BSA was used to measure the secondary structure changes. Both the spectra of BSA in the native state and in reconstituted solution showed a positive band around 190 nm and two negative bands around 208 nm and 222 nm, which indicates the predominantly α-helix structure of BSA, see FIG. 3A. Deconvolution of CD spectrum using Dichroweb showed the native and reconstituted BSA samples contained 57%-61% α-helix structure, which corresponds with known literature values [19, 24]. The protein degradation and/or aggregation can cause intensity change or peak position shift in the CD spectrum [23]. The results show that BSA maintained its secondary structure after spray layering, as the temperature of BSA on the surface of beads was lower than the product temperature which was controlled under 35° C. during the process due to evaporative cooling. After 38 days of storage at 40° C., as shown in FIG. 3B, a-helix structure decreased while β-strand and unordered structure increased. During this time, BSA kept its secondary structure when stored at 4° C. and 25° C. ; this data indicates the formulation has a degree of stability.

Tertiary structure determination—Derivative-UV spectrum of BSA was used to probe the tertiary structure change. The UV spectrum was collected from 200 nm to 400 nm using DU 800 spectrometer with an interval of 0.2 nm. The second derivative of the spectrum was calculated using Savitzky Golay algorithm in Origin 9.0 and fitted into cubic functions with a resolution of 0.02 nm for peak positions.

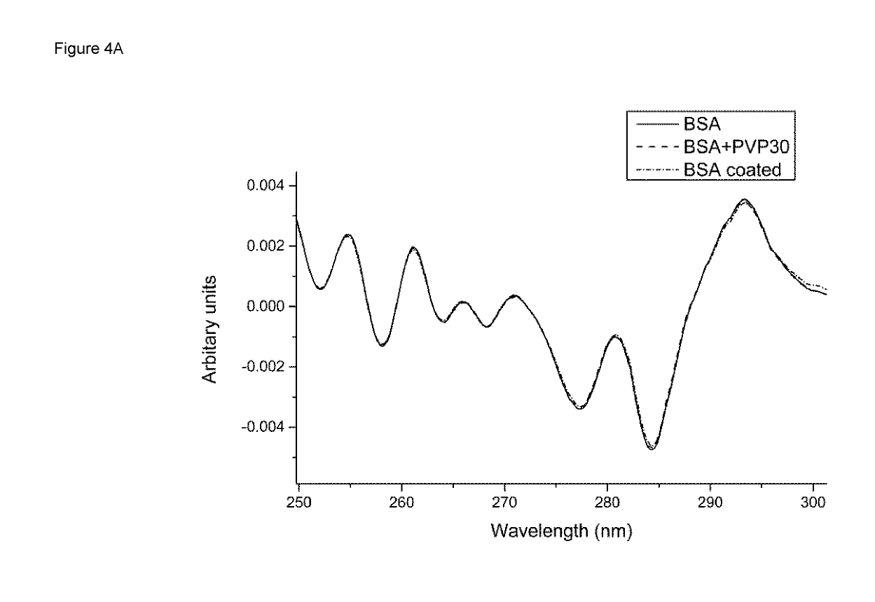

Derivative-UV spectrum of BSA was measured to probe any tertiary structure change. As shown in FIG. 4A, the peaks around 277 nm and 284 nm were assigned to tyrosine and the combination of tyrosine and tryptophan, respectively [25]. The position shift of these two peaks was believed to be sensitive to the micro-environment change of tyrosine and tryptophan [25]. As shown in FIG. 4A, the spectra of native BSA, BSA with PVP and BSA in the layered beads were nearly superimposable, detailed interpolation of the spectra revealed minor shift of the peak position from 277.08 nm to 277.32 nm, from 284.10 to 284.26 nm after spray layering. Although the blue shift was significant as shown by student t test, the shift was only 0.23 nm and 0.16 nm, and it is likely from the matrix effect that PVP decreased the solvent accessibility of BSA in solution. Additionally, a third peak around 290 nm detected and was likely attributed to tryptophan. After one month storage stability study, there was no big change in the peak around 290 nm detected in the 2^{nd} spectra of reconstituted BSA from 4° C. beads , 25° C. and 40° C. (FIG. 4B). A slight blue shift of two peaks was detected in the storage samples implying minimal tertiary structure differences.

Accelerated stability of BSA layered beads—ICH (The International Council for Harmonisation) accelerated stability conditions were used to test the stability of the BSA layered beads. Beads were stored in the 20 mL scintillation vials and purged with nitrogen before capped. Those vials were wrapped with aluminum foil and stored at three different conditions, 4° C., 25° C./60% relative humidity (RH) and 40° C./75% RH. Samples were taken out after 40 days and analyzed for aggregation, secondary structure and tertiary structure.

After a one month accelerated stability study, the insoluble aggregations (data not shown here) in samples reconstituted from that stored at 4° C. and 25° C. did not increase significantly, while those in the samples stored at 40° C. increased significantly (student t test, p=0.028, n=3). However, the soluble aggregation increased dramatically in samples both at 25° C. and 40° C. Further examination of CD spectra and UV spectra implies that even samples stored at 4° C. showed increased components of turns in the secondary structure, and increased peak intensity of 2^{nd} UV peak intensities at 277.2 nm and 284.2 nm. Although the perturbation at secondary and tertiary structure is deliberate, little research has been done to indicate that the small perturbation of protein structure could trigger long term storage issues for biologics [34].

### Enteric Coating of BSA Layered Beads

The BSA layered beads were further coated with an enteric coating layer comprising EUDRAGIT® FS 30 D in combination with 10% PlasACRYL™ T20 (based on dry polymer weight) as the plasticizer. The solid content of final solution was 20%. Coating with the enteric layer was conducted using the same fluid bed system with a nozzle size of 0.8 mm defined above. The inlet temperature was 35° C. and the product temperature was 25-28° C. The atomization pressure was 0.6 bar and the microclimate air pressure was 0.3 bar. The feed rate was 0.74 g/min. The coating thickness is 10%, 15%, 20% and 30% weight gain.

The enteric coating is usually conducted at a comparatively low temperature, for instance, the product temperature recommended for spraying aqueous dispersion of Eudragit FS30D is 25° C. to 30° C., which is below the first melting temperature of most therapeutic antibodies [26]. Thus, a change in protein structure or activity loss during the enteric coating process was not expected. The product temperature of 25-28° C. and inlet temperature of 35° C. was below the onset of unfolding temperature of BSA. Considering the evaporative cooling effects, the product temperature was much closer to the protein temperature during the process.

### In Vitro Release of BSA Beads

Previous studies on GI tract pH reported that the pH of the human intestinal tract increases from 1-2.5 (n=10) to 7.49 (n=58) in the ileum, drops to 6.37 (n=66) in the ascending colon, and increases to 7.04 (n=50) in the descending colon [27]. According to the United States Pharmacopeia (USP), the stomach simulated fluid is 0.1 N HCl with a pH of 1.2±0.05. Since the pH in intestine varies across human subjects, and may also be affected by fast state or disease states, there is no “gold standard” for simulated intestinal fluid or simulated colonic fluid. In this study, phosphate buffer with a pH of 6.8 and 7.4 was selected to simulate the intestinal and colonic fluid respectively [28-30]. The gastric volume varies from around 100 mL to more than 1000 mL due its distensibility, while the intestinal volume is approximately or lower than 100 mL for most cases [31-33]. The main point of this study was to show the protein release in the intestine especially in the colon, so the media to simulate the GI fluid was chosen as 100 mL buffer in a mini dissolution vessel.

The in vitro release of enteric coated beads was tested in a USP apparatus 1 with a vessel volume of 150 mL (Hanson Research, Chatsworth, Calif.). The media volume used was 100 mL. The media with pH 1.2, 6.8, and 7.4 were used to mimic the conditions of stomach, small intestine and large intestine, respectively. The dissolution test was performed at 37° C. and 100 rpm, kept 2 h in each media. The media was sampled every 30 mins and analyzed for protein concentration using UV-vis spectrometer at 280 nm. The sampling volume was compensated in calculation.

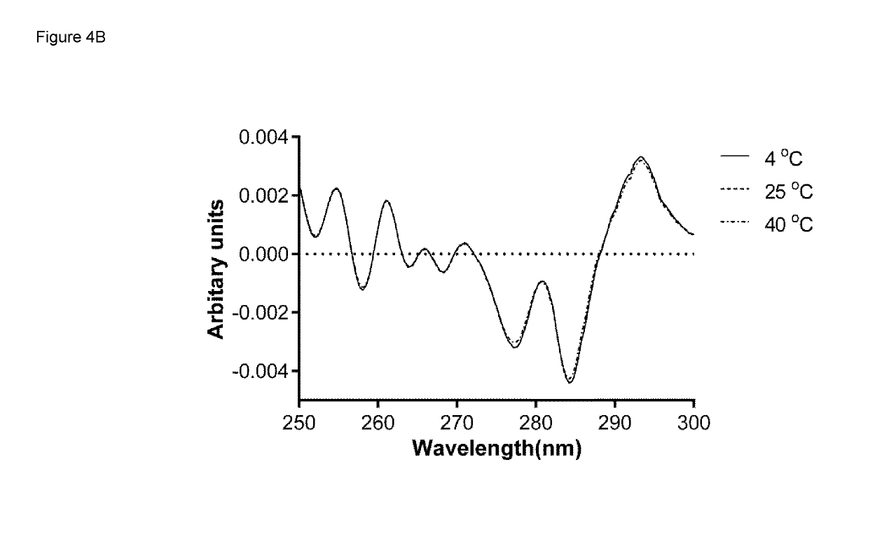

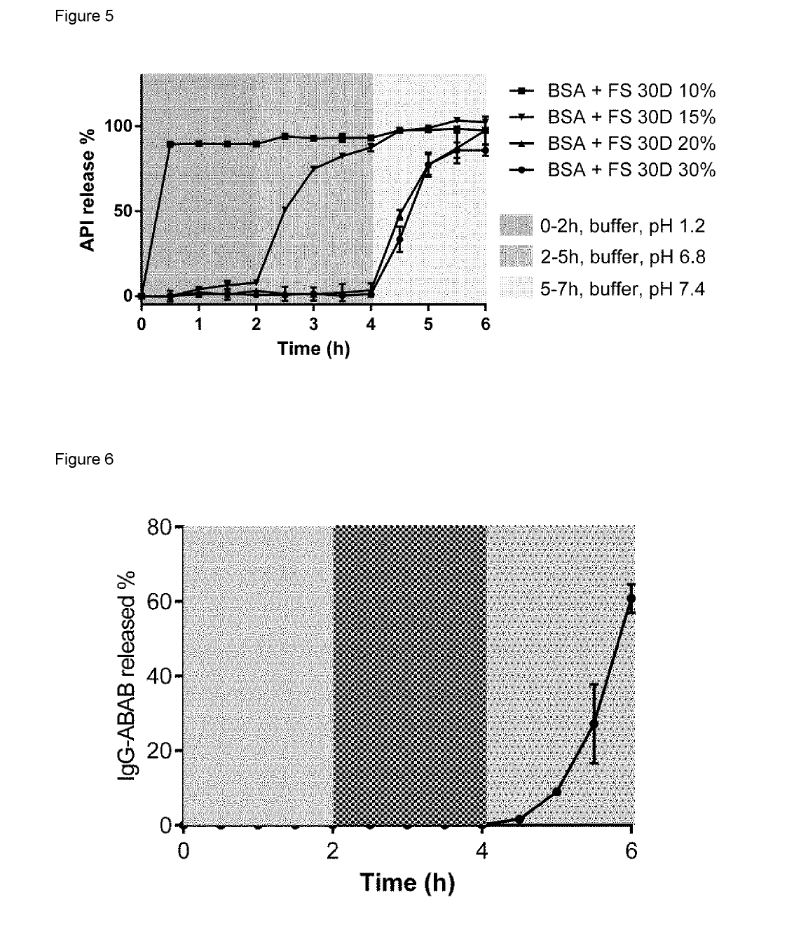

The coating thickness of enteric polymers was controlled by theoretical weight gain. As shown in FIG. 5, BSA layered microbeads coated with 10% Eudragit FS 30D released the BSA completely by 30 mins in acidic media, which indicates no intact film formed on the surface of beads. When the coating thickness increased to 15%, it showed partial protection as 7.9% of BSA was released by 2 hours in acidic media, and more than 87.5% of BSA released by 4 hours in a pH neutral media (pH 6.8). This might be due to the heterogeneity of films on different beads. The weight gain of 20% and 30% coatings showed complete protection for BSA since no BSA released in simulated gastric fluid (pH 1.2) and simulated small intestine fluid (pH 7.4). There was no significant difference of BSA release in 20% and 30% coating thickness based on f1 and f2 score.

### Example 2 —Preparation of Immunoglobulin Coated Microbeads

A proprietary antibody construct was used in place of BSA in the therapeutic agent layer. To create the antibody coated microbeads, a subcoat of PVP 30 (Kollidon 30) was first applied to D-mannitol beads to achieve 1% weight gain. The spray solution for the therapeutic agent layer was prepared as 1.85 mg/ml of immunoglobulin (termed “IgG-ABAB” herein; described in international patent publication WO 2016/127104, which is herein incorporated by reference in its entirety), and 2% (w/v) of PVP 30 in water. The spray coating was performed in Bosch Solidlab 1 fluid bed system. The fluid bed system was operated at the follow conditions: inlet air temperature=45° C., product temperature=35° C., atomization pressure=0.6 bar, microclimate air pressure=0.3 bar, air flow rate=10 m³/h. The feed rate was 0.74 g/min. A total of 100 mg of IgG-ABAB was sprayed onto the surface of 41 g of beads.

The IgG-ABAB layered beads were further coated with EUDRAGIT® FS 30 D as an enteric coating layer. PlasACRYL™ T20 was used as the plasticizer in conjunction with the EUDRAGIT. The enteric coating was conducted using Bosch Solidlab 1 fluid bed system with the follow parameters: inlet air temperature=35° C., product temperature=25° C. −29° C., atomization pressure=0.6 bar, microclimate air pressure=0.3 bar. The feed rate was 0.74 g/min. The coating thickness was a 30% weight gain.

The in vitro release of IgG-ABAB was tested using the sample apparatus and settings in defined above in Example 1. The sampled media were analyzed for IgG-ABAB concentration using an established ELISA method describe elsewhere. A representative in vitro dissolution profile is provided in FIG. 6, demonstrating that the immunoglobulin could be released from the microbeads over time.

### Example 3 —In Vivo Testing of Immunoglobulin Coated Microbeads

The in vivo release of IgG-ABAB was tested in a mouse model. Seven mice were fasted overnight, and placed into individual clean cages with water. The mice were divided into two groups (n=3 and n=4). Each mouse was given 0.4 g of cookie dough mixed with 0.1 g of the beads defined above in Example 2, dosed twice. The mice were sacrificed after 4 h. The GI tract was divided and collected. Each tissue sample was mixed with 500 uL of phosphate buffer (pH 6.5) and 100 times diluted protease inhibitor in 24 plates, gently shook for lh, centrifuged at 15000 rpm for 5 mins. The supernatants were measured for IgG-ABAB concentration using an established ELISA. The ELISA procedure included the following. 96 well plates were coated with 50 uL Clostridium difficile toxin B (0.5 ug/ml) per well overnight. The toxin was discarded and blocked with 100 uL 5% milk. The wells were washed, the samples added, and the plates incubated for 1 h at room temperature, before additional washed and addition of secondary antibodies. After incubating for an additional 1 h, the wells were washed, substrate was added, followed by stop solution. The plates were read at 450 nm.

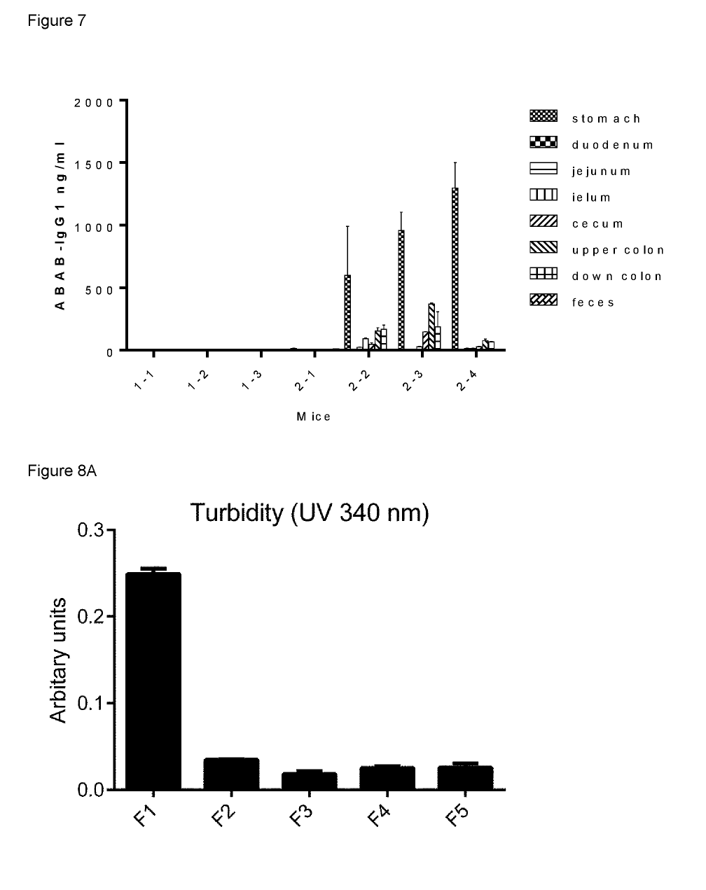

As shown in Tables 1 & 2 and FIG. 7, mice 1-1, 1-2, 1-3 were given BSA-coated beads, while mice 2-1, 2-2, 2-3, 2-4 were given Immunoglobulin (IgG-ABAB) coated beads. A large amount of Ig-ABAB was detected in the stomach, which was likely due to release from beads that were crushed when the mice chewed the cookie dough used to administer the beads. From duodenum to upper colon, a trend of increased concentration of IgG-ABAB was observed.

TABLE 1

| Mouse # | Stomach | | Duodenum | | Jejunum | | Ileum | |
|---|---|---|---|---|---|---|---|---|
| 1-1 | 0.72 | 0.72 | 0.29 | 0.35 | 0.29 | 0.35 | 0.35 | 0.23 |
| 1-2 | 2.3 | 1.97 | 0.17 | 0.29 | 0.35 | 0.59 | 0.23 | 0.29 |
| 1-3 | 0.23 | 0.23 | 0.23 | 0.23 | 0.29 | 0.29 | 0.17 | 0.17 |
| 2-1 | 17.07 | 13.12 | 0.35 | 0.17 | 0.17 | 0.17 | 0.17 | 0.17 |
| 2-2 | 875.46 | 321.73 | 0.29 | 0.29 | 22.1 | 24.73 | 87.33 | 97.17 |
| 2-3 | 1061.45 | 853.52 | 8.01 | 9.19 | 1.28 | 1.46 | 25.98 | 30.92 |
| 2-4 | 1441.76 | 1154.6 | 2.43 | 2.43 | 17.07 | 12.26 | 16.49 | 13.13 |

Values are expressed in ng/ml of IgG-ABAB

TABLE 2

| | | | | | Descending | | | |
|---|---|---|---|---|---|---|---|---|
| Mouse # | Cecum | | Upper Colon | | Colon | | Feces | |
| 1-1 | 0.35 | 0.23 | 0.29 | 0.29 | 0.35 | 0.35 | | |
| 1-2 | 0.35 | 0.29 | 2.3 | 2.62 | 0.29 | 0.35 | | |
| 1-3 | 0.17 | 0.23 | 0.17 | 0.47 | 0.23 | 0.17 | | |
| 2-1 | 6.08 | 5.87 | 0.41 | 0.41 | 0.41 | 0.29 | 11.14 | 11.14 |
| 2-2 | 57.02 | 36.44 | 136.28 | 173.35 | 143.51 | 192.07 | | |
| 2-3 | 148.95 | 145.5 | 376.6 | 365.35 | 102.02 | 271.62 | | |
| 2-4 | 31.51 | 24 | 85.41 | 70.12 | 69.93 | 65.78 | | |

Values are expressed in ng/ml of IgG-ABAB

This data shows that significant amount of IgG-ABAB was delivered to the lower part of GI tract in mice, especially the upper colon.

### Example 4 —Spray Layering of Therapeutic Agent Layer

In the production of the delivery vehicles of the invention, the therapeutic agent (e.g. a drug or an immunoglobulin) may be loaded onto the microbeads via spray layering. During a step of spray layering, formulation variables and process parameters will affect the final products, in terms of mass recovery, protein stability, etc. and only specific combinations will produce a useable product. In this example, it is shown how the formulation variables and process parameters can produce a microbead spray layered with an IgG immunoglobulin.

Five formulations of spraying solution for the therapeutic agent layer were studied for their effects on the microbeads after spray layering. Lyophilized human IgG powders purified from human plasma was purchased from Lee Biosolutions, Inc (Maryland Height, Mo.). Human IgG (10 mg/mL) was mixed with different excipients in spray layering formulations. The excipients studied included polyvinylpyrrolidone, trehalose, sucrose, L-arginine monohydrochloride. Mannitol microbeads (NONPAREIL®-108) were first sub-coated with 1% PVP 30 (other polymers are also suitable, e.g. HPMC, HPC, etc.) in a Bosch Mycrolab fluid bed system. The subcoated microbeads were then layered with one of the following protein formulations:
1. Formulation 1 (F1): IgG (10mg/mL) in water was used as a control
2. Formulation 2 (F2): IgG+PVP (2.5%)
3. Formulation 3 (F3): IgG+PVP (2.5%) +Trehalose (100% w/w of IgG)
4. Formulation 4 (F4): IgG+PVP (2.5%) +Sucrose (100% w/w of IgG)
5. Formulation 5 (F5): IgG+PVP (2.5%) +Arginine (100% w/w of IgG)

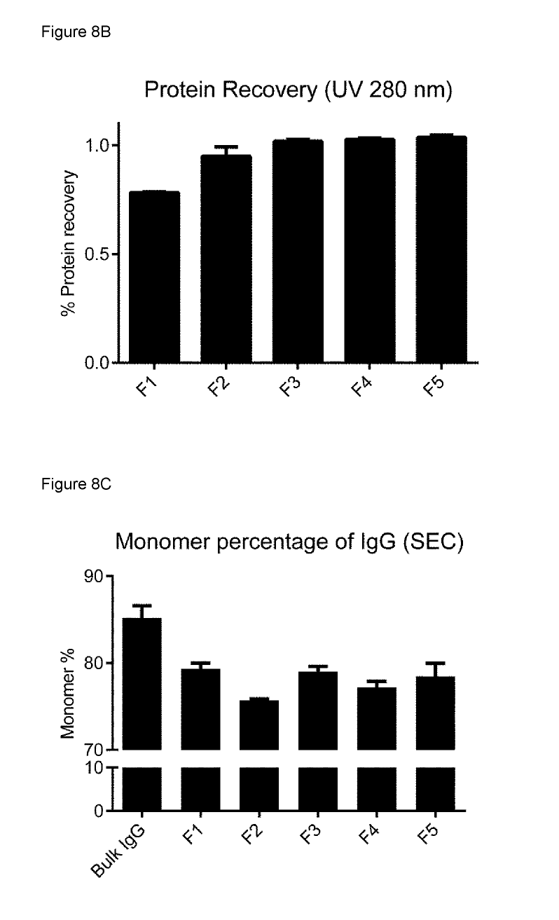

After the layering process, the spray layered beads were tested for stability by reconstituting in water. The reconstituted solution was measured for turbidity using a UV-vis spectrometer at a wavelength of 340 nm (see FIG. 8A). The protein recovery was measured by UV-vis spectroscopy at 280 nm using an extinction coefficient of 1.36 (see FIG. 8B). The monomer percentage of IgG in the reconstituted solution was measured by size-exclusion FPLC at 280 nm after filtration with a 0.22 micron polyethersulfone (PES) filter unit (see FIG. 8C). As shown in FIG. 8A, 8B and 8C, adding PVP to the formulation minimized the turbidity of IgG by 0.2 (arbitrary units) (compare F1 to F2-F5), while increasing the protein recovery by 19% after spray layering. This result showed that adding PVP lowered the insoluble aggregation of IgG and improved the adhesion of IgG to mannitol beads. Adding PVP alone resulted in an increase of soluble aggregation of IgG, while adding rehalose/sucrose/arginine stabilized the Ig.
To show the process conditions, the formulation containing PVP and trehalose (F3) was further studied. Such IgG layered beads were sieved to obtain a sieve cut (355-710 μm) to quantitate the process efficiency and then reconstituted in water for measurement of protein degradation. The turbidity of reconstituted solution was measured by UV-vis spectroscopy at 340 nm. The soluble aggregates of reconstituted samples after filtration (PES membrane, 0.22 micron) were measured by size exclusion chromatography (SEC). The binding activity of reconstituted IgG to anti-IgG antibody was measured using a standard enzyme-linked immunosorbent assay (ELISA). The list of process parameters studies are as follows:
1. Atomization pressure (AP), the nozzle pressure to atomize the spray solution
2. Feed rate (FR), the rate of protein formulation addition
3. Inlet temperature (IT), the temperature of inlet air into the fluid bed
4. Airflow rate (AR), the rate of inlet air into the fluid bed

The levels of these process parameters utilized in the study are provided in Table 3.

TABLE 3

| | Variable levels | | |
|---|---|---|---|
| AP (bar) | 0.4 | 0.6 | 0.8 |
| FR (rpm) | 9 | 12 | 15 |
| IT (° C.) | 45 | 55 | 65 |
| AR (m³/h) | 8 | 10 | 12 |

The five responses evaluated are as follows:
1. Process Efficiency: the total mass recovery=weight of solids (plus solids from spray solution) supplied into fluid bed/weight of solids recovered from fluid bed
2. Protein Recovery: protein sprayed/protein reconstituted
3. Turbidity: UV reading at 340 nm
4. Monomer Percentage: peak area of monomer peak/total peak areas
5. In vitro Binding Activity: binding affinity to anti-IgG antibodies measured by ELISA

Table 4 shows the results of the effects of spray layering process parameters on IgG properties. As seen in Table 4, the results showed no significant effects from the four process factors on the process efficiency and IgG recovery. The turbidity was affected by the interaction between inlet air temperature and air flow rate. All main factors except atomization pressure had significant effects on monomer percentage, among which air flow rate was the most significant. Only inlet air temperature had significant effects on the binding activity of IgG after spray layering.

TABLE 4

| | | | | | Binding |
|---|---|---|---|---|---|
| | Efficiency | Recovery | Turbidity | Monomer | Activity |
| AP | 6.11 | −1.33 | −0.009 | 0.21 | 0.57 |
| FR | −1.25 | −0.55 | 0.005 | 0.83* | −3.72 |
| IT | 5.78 | 3.28 | −0.002 | −0.86* | 18* |
| AR | 6.15 | 0.55 | −0.003 | −2.11* | −3.10 |
| AP*FR | −1.91 | −4.75 | −0.001 | −0.44 | 11.17 |
| AP*IT | 0.30 | 1.56 | −0.002 | 0.26 | 3.15 |
| AP*AR | −0.57 | 1.62 | −0.003 | 0.05 | −9.74 |
| FR*IT | −4.26 | −0.41 | 0.002 | 0.24* | −1.07 |
| FR*AR | −2.30 | −1.58 | 0.009 | 0.18 | 2.30 |
| IT*AR | −5.48 | −6.20 | 0.011* | −0.60* | −11.81 |

*indicates process results are statistically significant

Both formulation and process variables played important roles in the spray layering process of human IgG. By optimizing the formulation, recovery of almost 100% spray layered IgG and reduced IgG aggregation during the process were achieved. The study showed the combination of variables that produced layered beads that had good stability.

While the invention has been described with reference to certain particular embodiments thereof, those skilled in the art will appreciate that various modifications may be made without departing from the spirit and scope of the invention. The scope of the appended claims is not to be limited to the specific embodiments described.

### REFERENCES

All patents and publications mentioned in this specification are indicative of the level of skill of those skilled in the art to which the invention pertains. Each cited patent and publication is incorporated herein by reference in its entirety. All of the following references have been cited in this application:
1. Walsh, G., Biopharmaceutical benchmarks 2014. Nature biotechnology, 2014. 32(10): p. 992-1000.
2. Leader, B., Q. J. Baca, and D. E. Golan, Protein therapeutics: a summary and pharmacological classification. Nature reviews. Drug discovery, 2008. 7(1): p. 21-39.
3. Peniche, A. G., T. C. Savidge, and S. M. Dann, Recent insights into Clostridium difficile pathogenesis. Current opinion in infectious diseases, 2013. 26(5): p. 447-453.
4. Giesemann, T., et al., Processing of Clostridium difficile toxins. Journal of medical microbiology, 2008. 57(Pt 6): p. 690-6.
5. Abdul, S., A. V. Chandewar, and S. B. Jaiswal, A flexible technology for modified-release drugs: Multiple-unit pellet system (MUPS). Journal of Controlled Release, 2010. 147(1): p. 2-16.
6. Potesta, P., Eudragit FS 30 D: a new pH-sensitive polymer covering for mesalamine. European review for medical and pharmacological sciences, 2001. 5(1): p. 30.
7. Gao, C., et al., In vitro release and in vivo absorption in beagle dogs of meloxicam from Eudragit FS 30 D-coated pellets. International journal of pharmaceutics, 2006. 322(1-2): p. 104-12.
8. Kaledaite, R., et al., The development and in vitro evaluation of herbal pellets coated with Eudragit FS 30. Pharmaceutical development and technology, 2014: p. 1-6.
9. Weijers, R. N., Amino acid sequence in bovine serum albumin. Clinical chemistry, 1977. 23(7): p. 1361-2.
10. Wada, Y., Primary sequence and glycation at lysine-548 of bovine serum albumin. Journal of mass spectrometry : JMS, 1996. 31(3): p. 263-6.
11. Reed, R. G., F. W. Putnam, and T. Peters, Jr., Sequence of residues 400-403 of bovine serum albumin. The Biochemical journal, 1980. 191(3): p. 867-8.
12. Benjamin, D. C. and J. M. Teale, The antigenic structure of bovine serum albumin. Evidence for multiple, different, domain-specific antigenic determinants. The Journal of biological chemistry, 1978. 253(22): p. 8087-92.
13. Militello, V., V. Vetri, and M. Leone, Conformational changes involved in thermal aggregation processes of bovine serum albumin. Biophys Chem, 2003. 105(1): p. 133-41.
14. Huang, B. X., H. Y. Kim, and C. Dass, Probing three-dimensional structure of bovine serum albumin by chemical cross-linking and mass spectrometry. Journal of the American Society for Mass Spectrometry, 2004. 15(8): p. 1237-47.
15. Shanmugam, G. and P. L. Polavarapu, Vibrational circular dichroism spectra of protein films: thermal denaturation of bovine serum albumin. Biophys Chem, 2004. 111(1): p. 73-7.
16. Barreca, D., et al., Anti-aggregation properties of trehalose on heat-induced secondary structure and conformation changes of bovine serum albumin. Biophys Chem, 2010. 147(3): p. 146-52.

17. Majorek, K. A., et al., Structural and immunologic characterization of bovine, horse, and rabbit serum albumins. Molecular immunology, 2012. 52(3-4): p. 174-82.
18. Bhattacharya, M., N. Jain, and S. Mukhopadhyay, Insights into the Mechanism of Aggregation and Fibril Formation from Bovine Serum Albumin. J Phys Chem B, 2011. 115(14): p. 4195-4205.
19. Estey, T., et al., BSA degradation under acidic conditions: A model for protein instability during release from PLGA delivery systems. Journal of pharmaceutical sciences, 2006. 95(7): p. 1626-1639.
20. Bowen, M., R. Turok, and Y. F. Maa, Spray Drying of Monoclonal Antibodies: Investigating Powder-Based Biologic Drug Substance Bulk Storage. Dry Technol, 2013. 31(13-14): p. 1441-1450.
21. Mumenthaler, M., C. C. Hsu, and R. Pearlman, Feasibility Study on Spray-Drying Protein Pharmaceuticals-Recombinant Human Growth Hormone and Tissue-Type Plasminogen-Activator. Pharmaceutical research, 1994. 11(1): p. 12-20.
22. Kelly, S. M., T. J. Jess, and N. C. Price, How to study proteins by circular dichroism. Bba-Proteins Proteom, 2005. 1751(2): p. 119-139.
23. Joshi, V., et al., Circular Dichroism Spectroscopy as a Tool for Monitoring Aggregation in Monoclonal Antibody Therapeutics. Anal Chem, 2014. 86(23): p. 11606-11613.
24. Carter, D. C. and J. X. Ho, Structure of Serum-Albumin. Adv Protein Chem, 1994. 45: p. 153-203.
25. Mach, H. and C. R. Middaugh, Simultaneous monitoring of the environment of tryptophan, tyrosine, and phenylalanine residues in proteins by near-ultraviolet second-derivative spectroscopy. Analytical biochemistry, 1994. 222(2): p. 323-31.
26. Ionescu, R. M., et al., Contribution of variable domains to the stability of humanized IgG1 monoclonal antibodies. Journal of pharmaceutical sciences, 2008. 97(4): p. 1414-26.
27. Ashford, M. and J. T. Fell, Targeting drugs to the colon: delivery systems for oral administration. Journal of drug targeting, 1994. 2(3): p. 241-57.
28. Mura, P., et al., Development of enteric-coated pectin-based matrix tablets for colonic delivery of theophylline. Journal of drug targeting, 2003. 11(6): p. 365-71.
29. Alvarez-Fuentes, J., et al., Development of enteric-coated timed-release matrix tablets for colon targeting. Journal of drug targeting, 2004. 12(9-10): p. 607-12.
30. Cole, E. T., et al., Enteric coated HPMC capsules designed to achieve intestinal targeting. International journal of pharmaceutics, 2002. 231(1): p. 83-95.
31. Ong, B. Y., R. J. Palahniuk, and M. Cumming, Gastric volume and pH in out-patients. Canadian Anaesthetists' Society journal, 1978. 25(1): p. 36-9.
32. Ferrua, M. and R. Singh, Modeling the fluid dynamics in a human stomach to gain insight of food digestion. Journal of food science, 2010. 75(7): p. R151-R162.
33. Schiller, C., et al., Intestinal fluid volumes and transit of dosage forms as assessed by magnetic resonance imaging. Alimentary pharmacology & therapeutics, 2005. 22(10): p. 971-9.
34. Park J, Nagapudi K, Vergara C, Ramachander R, Laurence J S, Krishnan S. Effect of pH and excipients on structure, dynamics, and long-term stability of a model IgG1 monoclonal antibody upon freeze-drying. Pharmaceutical research, 2013. 30: p. 968-84.

1. A delivery vehicle comprising a microbead coated with a subcoat layer, a therapeutic agent layer, and an enteric coating layer,
wherein the microbead comprises one or more physiologically inert substances;
wherein the subcoat layer substantially coats the microbead;
wherein the therapeutic agent layer substantially coats the subcoat layer, and wherein the therapeutic agent layer comprises a therapeutic agent and an excipient; and
wherein the enteric coating layer substantially coats the therapeutic agent layer, and wherein the enteric coating layer comprises a pH-resistant composition.

2. The delivery vehicle of claim 1, wherein the microbead comprises one or more of a sugar, a starch, microcrystalline cellulose (MCC), a biodegradable polymer, sodium phosphate, and calcium phosphate.

3. The delivery vehicle of claim 2, wherein the sugar is selected from the group consisting of lactose, sucrose, mannitol, trehalose, maltodextrin, dextrose, fructose and a polysaccharide.

4. The delivery vehicle of claim 2, wherein the biodegradable polymer is poly(lactic-co-glycolic acid) (PLGA) or a copolymer of L-lactic acid and glycolic acid.

5. The delivery vehicle of claim 1, wherein the microbead further comprises a medicament.

6. The delivery vehicle of claim 1, wherein the microbead has an average particle diameter of between 1 and 1000 μm.

7. The delivery vehicle of claim 1, wherein the subcoat layer comprises one or more of ammonium alginate, cellaburate, chitosan, colophony, copovidone, ethylene glycol and vinyl alcohol grafted copolymer, gelatin, hydroxypropyl cellulose, hypromellose, hypromellose acetate succinate, polymethacrylate, poly(methyl vinyl ether/maleic anhydride), polyvinyl acetate dispersion, polyvinyl acetate phthalate, polyvinyl alcohol, polyvinylpyrrolidone (PVP), povidone, pullulan, pyroxylin, and shellac.

8. The delivery vehicle of claim 1, wherein the subcoat layer provides an amount of weight gain to the microbeads of between about 0.1 and 5%.

9. The delivery vehicle of claim 1,
wherein the subcoat layer comprises PVP and imparts a 1% weight gain to the microbeads.

10. The delivery vehicle of claim 1, wherein the therapeutic agent comprises one or more of a protein, a peptide, an antibody, an antiviral, an antifungal, an antibiotic, an anticancer agent, an analgesic, an anticoagulant, an antidepressant, an antiepileptic, an antipsychotic, and a sedative.

11. The delivery vehicle of claim 10, wherein the therapeutic agent is an antibody.

12. The delivery vehicle of claim 10, wherein the therapeutic agent is an antibody or fragment thereof having binding specificity for C. difficile toxin A and/or toxin B.

13. The delivery vehicle of claim 1, wherein the excipient comprises one or more of polyvinylpyrrolidone (PVP), polyethylene glycol (PEG), polyvinyl acetate (PVA), hydroxypropyl cellulose (HPC), sucrose, trelose, acacia, tragacanth, gelatin, starch, pregelatinized starch, alginic acid, cellulose, methyl cellulose, ethyl cellulose, sodium carboxy methyl cellulose, polymethacrylate, asparagine, dextran, glycine, inulin, lactose, anhydrous lactose, monohydrate, mannitol, raffinose, and trehalose.

14. The delivery vehicle of claim 1, wherein the therapeutic agent layer provides an amount of weight gain to the microbeads comprising the subcoat layer of between about 10 and 20%.

15. The delivery vehicle of claim 1, wherein the pH-resistant composition of the enteric coating layer comprises one or more of inulin, shellac, methacrylated inulin, pectin, chitosan, Eudragit® FS 30D, Eudragit® S 100 and Eudragit® S12,5.

16. The delivery vehicle of claim 15, wherein the pH-resistant composition of the enteric coating layer further comprises a plasticizer.

17. The delivery vehicle of claim 1, wherein the enteric coating layer provides an amount of weight gain to the microbeads comprising the subcoat layer and the therapeutic agent layer of between about 20 and 30%.

18. The delivery vehicle of claim 1, wherein the microbead is further coated with a sustained release layer, and wherein the sustained release layer substantially coats the therapeutic agent layer, and wherein the enteric coating layer substantially coats the sustained release layer.

19. The delivery vehicle of claim 18, wherein the sustained release layer comprises one or more of acacia, agar, alginic acid, aliphatic polyester, calcium alginate, carbomer, carrageenan, cellaburate, cellulose acetate, ceratonia, copovidone, gellan gum, guar gum, hydroxyethylmethyl cellulose, hydroxypropyl betadex, hydroxypropyl cellulose, hypromellose, methylcellulose, polycarbophil, poly(DL-lactic acid), polymethacrylate, polyoxylglyceride, polyvinyl acetate dispersion, shellac, sodium alginate, starch modified, xanthan gum, zein, Eudragit® RL, Eudragit® RL 30D, Eudragit® RL PO, Eudragit® RL 100, Eudragit® RL 12,5, Eudragit® RS 30 D, Eudragit® RS PO, Eudragit® RS 100, Eudragit® RS 12,5, Eudragit® NE 30 D, Eudragit® NE 40 D, and Eudragit® NM 30 D.

20. The delivery vehicle of claim 18, wherein the sustained release layer provides an amount of weight gain to the microbeads comprising the subcoat layer and the therapeutic agent layer of between about 5 and 15%.

21-54. (canceled)
